(19) United States
(12) Patent Application Publication
(10) Pub. No.: US 2025/0346848 A1
(43) Pub. Date: Nov. 13, 2025

(54) Energy Efficient Production Process

(71) Applicant: The Protein Brewery B.V., Breda (NL)

(72) Inventors: Johannes Jacobus Joseph Den Ridder, Huijbergen (NL); Wilhelmus Theodorus Antonius Maria De Laat, Breda (NL)

(73) Assignee: The Protein Brewery B.V., Breda (NL)

(21) Appl. No.: 18/870,475

(22) PCT Filed: May 31, 2023
(86) PCT No.: PCT/EP2023/064537

§ 371 Date: Nov. 29, 2024

(30) Foreign Application Priority Data
Jun. 3, 2022 (NL) 2032074

(51) Int. Cl. C12M 1/02; A23J 1/00; C12M 1/00; C12M 1/34; C12P 21/00

(52) U.S. Cl. CPC C12M 41/20; A23J 1/008; C12M 21/02; C12M 41/26; C12M 47/10; C12P 21/00

(57) ABSTRACT

The invention relates to a process for reusing heat in a protein culture, the process comprising the steps of aerobically fermenting a material with an thermophilic organism to provide a thermophilic fermented culture; and performing a drying process on the thermophilic fermented culture using heat produced during the aerobic fermentation. The invention further relates to a system for reusing heat in a protein culture.

### FIELD OF THE INVENTION

The invention relates to an energy efficient production process, particularly a process for reusing heat in a protein culture. The invention further relates to a system for reusing heat in a protein culture.

### BACKGROUND

The increasing global population leads to rapidly increasing demand for protein-rich food like meat, dairy products, insects and fish. One method for producing protein-rich animal feed is to produce single cell protein (SCP) by means of biosynthesis processes, as fermentation.

Such biosynthesis processes are powered up by traditional energy sources, such as, for example, fossil fuel. Fossil fuels are non-renewable resources because they take millions of years to form. Furthermore, the use of fossil fuels raises serious environmental concerns. Renewable energy sources, such as biomass and solar, represent a promising alternative to many traditional energy sources. However, scaling up of such renewable energy source poses problems with energy demand and productions costs.

It is an object of the present invention to address these problems in the production of single cell protein.

### SUMMARY

According to an embodiment, the present application provides a process for reusing heat in a protein culture, the process comprising the steps of aerobically fermenting a material with an thermophilic organism to provide a thermophilic fermented culture; and performing a drying process on the thermophilic fermented culture using heat produced during the aerobic fermentation. This provides extra energy in the drying process, which will result in a reduction or minimization of external energy consumption for driving the drying process. As a result, the re-use of energy increases the efficiency of the overall process and saves on overall energy usage to help with the challenges of climatic change and limiting the use of fossil fuel resources.

Furthermore, heat or part of the heat produced during fermentation is reused, which decreases the heat expelled to the environment. This could significantly reduce thermal pollution, a part of avoiding the use of a cooling system.

According to an embodiment, the process further comprises using a first heat exchanger to capture the heat produced during the aerobic fermentation process and heating an air flow for the drying process. Heat exchangers avoid any form of combustion heating, thereby reducing or eliminating pollution and risk of carbon monoxide and/or carbon dioxide poisoning.

According to an embodiment of the invention, the first heat exchanger is a heat pump. By using a heat pump, other than the electricity needed to power the pump, they consume no fuel, so they produce little or no carbon emissions, thereby reducing the carbon footprint. Consequently reducing greenhouse gas emissions. Optionally, the heat pump uses ammonia or R1234 as a refrigerant.

According to an embodiment, the process further comprises increasing the temperature of the air flow flowing out of the first heat exchanger using a second heat exchanger. By further using the second heat exchanger, the temperature of the air flow used in the drying process can be increased above 100 C. Optionally, the second heat exchanger is a steam heat exchanger. Optionally, the second heat exchanger includes one or more steam heat exchangers and one or more heat pumps.

According to an embodiment, capturing the heat produced during aerobic fermentation comprises capturing heat from a gas flow discharged during the fermentation. By using the heat produced during the aerobic fermentation, an additional source of energy to heat an air flow to be used in the drying process is avoided. Optionally, the gas flow has a temperature of 36 C or more. Preferably, the gas flow has a temperature of 38 C or more. More preferably, the gas flow has a temperature between 46 C and 54 C. Optionally, the gas flow has a relative humidity of 100%.

According to an embodiment, the heated air flow used during drying has a temperature of 70 C or more, for example 75 C, 80 C, 90 C, or even 100 C or 120 C. This temperature of 70 C or more ensures that the fermented culture dries at required conditions in order to obtain a protein culture. Using a temperature of the air flow during drying between 70 C and 90 C is more optimal than using a temperature of 100 C in terms of obtaining the protein culture.

According to an embodiment, the drying process comprises drying, in a first step, the thermophilic fermented culture using the heated air flow, and drying, in a second step, the thermophilic fermented culture using heat produced during the first step of drying. By using a multi-step drying process an exhaust air of a dryer is heated again and reused for a next drying step, the energy required for heating an air flow is reduced as the drying can be performed in several steps, each step using a different temperature for drying. This provides extra energy in the drying process, which will result in a reduction or minimization of external energy consumption for driving the drying process.

According to an embodiment, using the heat produced during the first step of drying comprises using a third heat exchanger to capture the heat from a first air flow discharged during the first step of drying and heating a second air flow. Optionally, the third heat exchanger is a heat pump.

According to an embodiment, the process further uses the heat produced during the aerobic fermentation in a clean-in-process, CIP.

According to an embodiment, sterile air is introduced during the aerobic fermentation. Introducing sterile air can prevent the invasion of foreign fungal spores or yeasts. Optionally, the sterile air is introduced at a predetermined air flow rate. By introducing air at a predetermined flow rate, it is ensured that fermentation continues without stopping. Optionally, the predetermined air flow rate is between 0,5 and 2,0 liquid volumes per minute (vvm).

According to an embodiment, at least one substance is introduced during aerobic fermentation to control pH during fermentation. The at least one substance is, for example, ammonia (NH₃), sulfuric acid (H₂SO₄), phosphoric acid (H₃PO₄), NaOH, nitrite hydrate (H2NO3), and nitrogen sources ammonium phosphate ((NH₄)₃PO₄), diammonium sulfate (NH₄)₂SO₄), urea (CH₄N₂O) and the like.

According to an embodiment, the present application provides a system for reusing heat in a protein culture, the system comprising: a fermenter configured to: aerobically ferment a material with an thermophilic organism to provide a thermophilic fermented culture, and discharge a gas flow out of the fermenter; a dryer configured to: dry the thermophilic fermented culture to recover a protein culture; and a heat exchange system configured to: capture heat from the discharged gas flow of the fermenter and use the captured heat to provide a hot gas flow for the dryer.

According to an embodiment, the fermenter comprises an air inlet through which sterile air may be introduced. Optionally, the sterile air is introduced at a predetermined air flow rate.

According to an embodiment, the fermenter comprises a second air inlet through which at least one substance to control pH may be introduced. Optionally, the air inlet through which sterile air is introduced, and the second air inlet through which at least one substance is introduce can be the same air inlet.

According to an embodiment, the system further comprises a second heat exchanger configured to further increase the temperature of the heated air flow used in the dryer. Optionally, the second heat exchanger is a steam heat exchanger. Optionally, the second heat exchanger is one or more steam heat exchangers and one or more heat pumps.

According to an embodiment, the system further comprises a first dryer configured to dry the thermophilic fermented culture by using the heated air flow; a third heat exchanger configured to capture heat from a first air flow discharged out of the first dryer and use the captured heat to provide a heated second air flow; and a second dryer configured to dry the thermophilic fermented culture to recover a protein culture by using the second air flow. Optionally, the third heat exchanger is a heat pump.

According to an embodiment, the system further comprises a condenser to heat up process water, cleaning water or heating an office or brewery building.

According to an embodiment, the system further comprises a CIP system configured to use heat produced during the aerobic fermentation.

### BRIEF DESCRIPTION OF THE DRAWINGS

The invention will be described further with respect to embodiments shown in the drawings.

FIG. 1 shows a flowchart illustrating a process for reusing heat in a protein culture according to an embodiment of the invention.

FIG. 2 shows a schematic representation of a system for reusing heat in a protein culture according to an embodiment of the invention.

FIG. 3 shows a schematic representation of a system for reusing heat in a protein culture according to another embodiment of the invention.

FIG. 4 shows a schematic representation of a system for reusing heat in a protein culture according to another embodiment of the invention.

FIG. 5 shows a schematic representation of a drying process according to another embodiment of the invention.

FIG. 6 shows an exemplary representation of a drying process using a dryer.

### DESCRIPTION

The following is a description of certain embodiments of the invention, given by way of example only and with reference to the figures. It is also to be understood that the terminology used herein is for the purpose of describing particular aspects only and is not intended to be limiting.

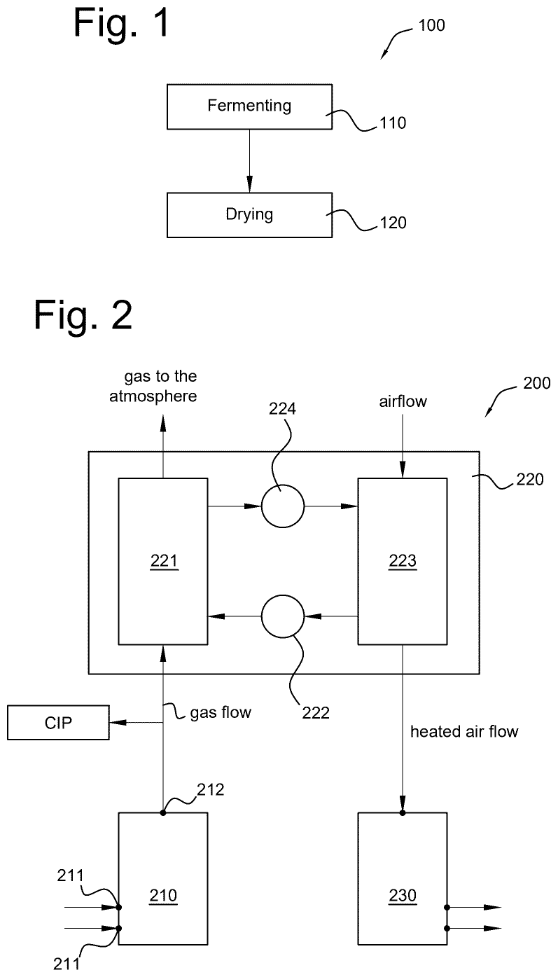

FIG. 1 shows a flowchart illustrating a process 100 for reusing heat in a protein culture according to an embodiment.

In an initial step 120 the process 100 comprises aerobically fermenting a material with an thermophilic organism to provide a thermophilic fermented culture.

The thermophilic organism for use in the invention refers to an organism that grows at a temperature of at least 36, 37, 38, 39, 40, 45, 50 or 55 C, sometimes even higher than 65 C. The thermophilic organism for use further refers to an organism that also grows at low pH, e.g., at a pH of 4.4 or less. The thermophilic organism for use further refers to an organism from which biomass can be obtained with a high protein content. The material for use is a feedstock that can serve as carbon and energy source for the thermophilic organism.

Fermentation is understood in this context as the conversion of a material (e.g. feedstock) into a product (e.g. protein culture) by using an organism (e.g. a thermophilic organism). During fermentation gas, as CO₂, water and heat are also produced. Traditionally, there are two types of fermentation: aerobic fermentation, which requires oxygen, and anaerobic fermentation, which does not require oxygen.

Optionally, the process 100 may comprise introducing air during fermentation. The air introduced during fermentation can comprise oxygen at a concentration above 0.2 mg/L in order to optimize growth conditions. Optionally, the oxygen concentration can be above 0.5 mg/L, or above 1 mg/L or above 2 mg/L. Optionally, the air can be introduced at a predetermined air flow rate. For example, the predetermined air flow rate is between 0.5 and 2.0 liquid volumes per minute (vvm). For example, 0.5 vvm, 1 vvm, 1.5 vvm or 2.0 vvm. The measurement unit “vvm” is calculated by dividing measured airflow rate (L/m) with the volume (L) of growth medium (including cultured cells). Alternatively, the air can be introduced at a variable air flow rate which may depend on fermenting conditions. Optionally, the air introduced during fermentation can be sterilized or unsterilized. Sterilisation of air can help to prevent fungal spores and yeasts and bacteria invading the fermentation.

Optionally, the process 100 may comprise introducing at least one substance to control pH during fermentation. The at least one substance is, for example, ammonia (NH₃), sulfuric acid (H₂SO₄), phosphoric acid (H₃PO₄), NaOH, nitrite hydrate (H2NO3), and nitrogen sources ammonium phosphate ((NH₄)₃PO₄), diammonium sulfate (NH₄)₂SO₄), urea (CH₄N₂O) and the like.

As shown in FIG. 1, in step 120, the process 100 comprises performing a drying process on the thermophilic fermented culture using heat produced during the aerobic fermentation.

During drying, moisture content of the thermophilic fermented culture is reduced to obtain a protein culture through the application of heat via a warm air flow. Since aerobic fermentation produces large amounts of heat (as opposed to anaerobic fermentation where the heat production is much lower due to lack of airflow through), the heat produced during the aerobic fermentation can be used to heat an air flow for use in the drying process without an additional source of energy. In case of anaerobic fermentation, an additional source of energy is typically needed to power the dryer, as consequence, is more costly and less environmentally friendly.

Optionally, the process 100 comprises using a first heat exchanger to capture the heat produced during the aerobic fermentation process and heating an air flow for the drying process. For example, the temperature of the heated air flow is 70 C or higher up to 100 C, which may correspond to the temperature required for the drying process. As another example, the temperature of the heated air flow is 50 C, which may be lower than the temperature required for the drying process.

In the context of the present application, “heat exchanger” means any apparatus that is useful for transferring heat from one medium to another. These media may be a gas, liquid, or a combination of both. Different types of heat exchangers can be used, for example, a heat pump, a heat pipe exchanger, a steam heat exchanger, a recuperator, and the like. Preferably, the heat exchanger is a heat pump and/or a steam heat exchanger. By using a steam heat exchanger instead of a heat pump, the increasing of the temperature above 100 C is cheaper.

A heat pump can comprise an evaporator, a compressor, a condenser and an expansion device. Refrigerant circulating in the evaporator at a low pressure captures heat from warm air flowing through the evaporator. As the refrigerant absorbs heat, the refrigerant passes from a liquid state to a vapour state. The refrigerant in the vapour state is compressed to a high pressure in the compressor. By compressing the refrigerant, the pressure and temperature of the refrigerant increase. The refrigerant then circulates in the condenser where an air flow is flowing, usually colder than the refrigerant. The air flow captures heat from the refrigerant, such that the refrigerant condenses. As the refrigerant releases heat, the refrigerant passes from the vapour state back to the liquid state. Afterwards, the refrigerant in the liquid state passes from a high pressure to a low pressure in the expansion device. The refrigerant flows again to the evaporator starting the cycle again.

The refrigerant typically for used is a natural refrigerant or a synthetic refrigerant. Examples of natural refrigerant are ammonia, carbon dioxide (CO₂), hydrocarbons-—as butane and isobutane—, water and air. Examples of synthetic refrigerant are (hydro) chlorofluorocarbon ((H)CFC), hydrofluorocarbon (HFC) and hydrofluoroolefin (HFO). Optionally, the refrigerant is ammonia. Ammonia has excellent thermodynamic properties, high heat transfer coefficients and does not contribute to the greenhouse effect. Furthermore, ammonia is inflammable and toxic but due to its strong odour, leakages can be detected fast. Alternatively, the refrigerant is R1234. R1234 is a hydrofluoroolefin refrigerant that does not contribute to the greenhouse effect. Although, R1234 has a low efficiency, it is non-flammable or only mildly flammable. Thus, R1234 supposes a lower cost inversion than ammonia due to its toxicity which may requires extra safety costs.

The efficiency of a heat pump is expressed as a coefficient of performance (COP). The COP is defined as a ratio between the rate at which the heat pump transfers thermal energy (in kW), and the amount of electrical power required to do the pumping (in kW). For example, if a heat pump used 1 kW of electrical energy to transfer 3 KW of heat, the COP would be 3. The higher the COP, the more efficient a heat pump is and the less energy it consumes.

A steam heat exchanger can comprise a shell enclosing a tube or tube bundle. The steam heat exchanger utilizes a steam which enters the shell through an opening and surrounds the tubes inside of the shell. As the latent heat of steam is transferred to a medium (e.g. an air flow) inside of the tubes, the temperature of the medium increases and condensation of the steam occurs. The condensate is then collected in the bottom of the shell and drained towards a condensate outlet. A steam trap is installed in the condensate outlet. The steam trap function is to hold the steam inside the shell until latent heat is transferred, and to drain the condensate once latent heat is transferred.

Optionally, a cleaning substance (e.g. water) can capture heat produced during fermentation to be used in a clean-in-place (CIP) process. CIP is a process for cleaning the interior surfaces of containers such as pipes, fermenters, vessels, condensers, and filters, without having to disassemble them, by circulating the cleaning substance(s) to clean and rinse interior surfaces. Typically, a final hot water rinse is performed to disinfect interior surfaces. By using heat produced during fermentation in the CIP process, to increase the temperature of the cleaning substance, the overall process becomes more efficient as none or few external energy is needed for heating the cleaning substance. Optionally, water can capture heat produced during fermentation to be used for other processes, such as for heating a building, for a wash water process, and the like, which require water heated above 45 C, or above 60 C up to 85 C.

Optionally, the process 100 comprises increasing the temperature of the heated air flow by using a second heat exchanger. In case the temperature of the air flow after being heated by the heat exchanger is lower than the required temperature for the drying process, the second heat exchanger may be used to further increase the temperature to the required temperature of the drying process. For example, in case the temperature of the heated air flow after flowing through the first heat exchanger is lower than the temperature required for the drying process, e.g. 50 C, the temperature can be further increased to, e.g., 70 C, 100 C or 120 C, using the second heat exchanger. Optionally, the second heat exchanger is a steam heat exchanger. Optionally, the second heat exchanger is one or more steam heat exchangers and one or more heat pumps.

Optionally, the drying process is a multi-step drying process. In a first step of the multi-step drying process, the heated air flow heated by the first heat exchanger (e.g. a first heat pump) using heat produced during the aerobic fermentation is used for drying the thermophilic fermented culture. In a second step, a temperature of an air flow discharged during the first step of the drying process is increased using a second heat exchanger (e.g. a steam heat exchanger) and this heated air flow heat is used for drying the thermophilic fermented culture.

Optionally, the first heat pump of the process 100 may comprise another condenser for increasing a temperature of process water. Process water can capture heat produced during fermentation to be used for other processes, such as for heating a building, for a wash water process, and the like, which require water heated above 45 C, or above 60 C up to 85 C.

Hereinafter, and for convenience, FIGS. 2-4 are described such that the first heat exchanger is a heat pump and the second heat exchanger, if present, is a steam heat exchanger. However, the skilled person would understand that any other heat exchanger can be used without departing from the scope of the invention.

FIG. 2 shows a schematic representation of a system 200 for reusing heat in a protein culture. The system 200 includes a fermenter 210, a first heat exchanger 220 and a dryer 230.

In the context of the present application, “fermenter” means any apparatus that is useful for growing organisms (such as yeast, fungi, bacteria, or animal cells) under controlled conditions.

The fermenter in which the processes described herein are run can be any type of fermenter known in the art, preferably, a fermenter suitable for aerobic fermentation. Advantageously the fermenter is a simple bubble column, which can be operated at very large scale such as e.g. >100 m³,>500 m³,>1000 m³,>2000 m³ or >3000 m³ or >4000 m³, thereby reducing the number of fermenters per factory, the total investment and operational cost.

In the present context, “dryer” means any apparatus that is useful for the reduction of the moisture content of a particulate material through the application of direct or indirect heat, including but not limited to a fluidized bed dryer, vibratory fluidized bed dryer, fixed bed dryer, traveling bed dryer, belt dryer cascaded whirling bed dryer, elongated slot dryer, hopper dryer, or kiln. Such dryers may also consist of single or multiple vessels, single or multiple stages, be stacked or unstacked, and contain internal or external heat exchangers.

The fermenter 210 includes at least one inlet 211 and at least one outlet 212. The fermenter may include more components such as sensors.

A thermophilic organism is provided into the fermenter in order for the aerobic fermentation of the material to occur. For aerobic fermentation sterile air flow is introduced into the fermenter 210 through the inlet 211.

During aerobic fermentation, a gas flow is exhausted from the fermenter 210 through the outlet 212. Since a thermophilic organism is used for fermentation, the fermenter 210 can be operated without any internal cooling system, not an internal cooling coil in the fermenter, cooling coil in baffles of a stirred fermenter or in the fermenter wall, no Riesel cooling or a cooling tower is required. An external cooling loop using a heat exchanger is not needed either. This will reduce the investment required, as the cooling will be done by evaporation of water, which will leave the fermenter via the gas flow exhaust of the fermenter 210 through the outlet 212. Optionally an external cooling jacket can be wrapped around the fermenter for helping to maintain the temperature during aerobic fermentation.

Preferably, the gas flow has 100% relative humidity. The 100% relative humidity indicates that that the gas is totally saturated with water vapor and cannot hold any more. Preferably, the gas flow has a temperature of 36 C or more. More preferable, between 36 Cand 65 C. Even more preferably, the gas flow has a temperature between 46 C and 50 C.

The first heat exchanger 220 of the system 200 is a heat pump 220 which includes an evaporator 221, a compressor 222, a condenser 223 and an expansion device 224.

The gas flow exhausted of the fermenter 210 through the outlet 212 flows through the evaporator 221 which captures the heat from the gas flow to heat a refrigerant. The heat absorbed by the refrigerant is later captured by an air flow entering into the condenser 223 such that the air flow is heated.

The heated air flow is then flowed through the drier 230 such that a protein culture is obtained from the fermented culture.

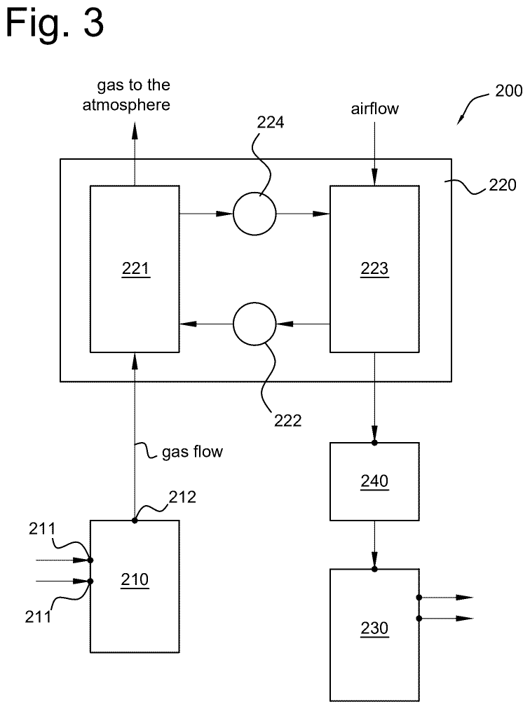

FIG. 3 shows a schematic representation of the system 200 for reusing heat in a protein culture in which the system 200 further includes a second heat exchanger 240.

During aerobic fermentation, the gas flow is exhausted from the fermenter 210 through the outlet 212. The first heat exchanger 220 of the system 200 is a heat pump 220 which includes an evaporator 221, a compressor 222, a condenser 223 and an expansion device 224. The gas flow exhausted from the fermenter 210 through the outlet 212 flows through the evaporator 221 which captures the heat from the gas flow to heat a refrigerant. The heat absorbed by the refrigerant is later captured by an air flow entering into the condenser 223 such that the air flow is heated.

The temperature of the heated air flow, e.g. 50 C, after flowing through the first heat exchanger 220 is lower than the temperature required for the drying process, e.g., 70 C. Hence, the temperature is further increased to, e.g., 120 C, by using the second heat exchanger 240 (e.g. a steam heat exchanger). Then, the heated air is flowed through the drier 230 such that a protein culture is obtained from the fermented culture.

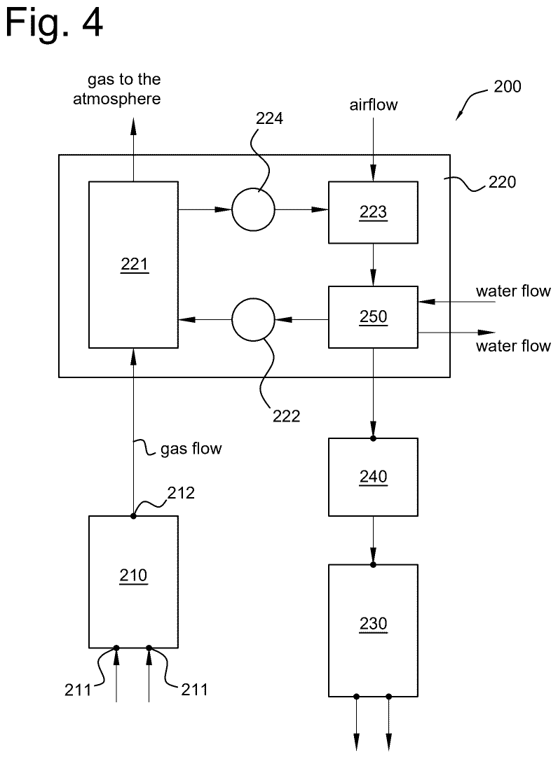

FIG. 4 shows a schematic representation of a system 200 for reusing heat in a protein culture in which the system 200 further includes an additional condenser 250 in the first heat exchanger 220.

By using the additional condenser 250, a water flow can capture heat produced during fermentation. The water flow circulates through the additional condenser 250 such that the temperature of the water flow is increased, for example, from 15 C to 50 C. The heated water flow can then be used for other processes, such as for heating a building, for a wash water process, and the like.

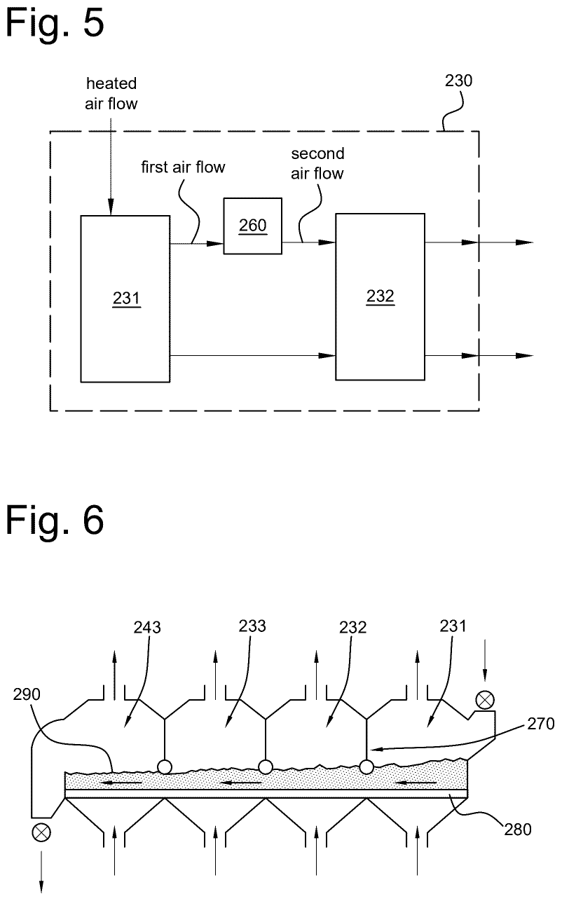

FIG. 5 shows a schematic representation of a drying process using a dryer. The dryer can be the dryer 230 of FIGS. 2-4. In FIG. 5, the dryer 230 comprises a first dryer 231 and a second dryer 232. The first dryer and the second dryer can refer to a first and second drying chamber, respectively. Optionally, the dryer 230 may comprise more than two dryers. In a first step of the drying process, the thermophilic fermented culture is dried using the air flow heated using the first heat exchanger 220, and the second heat exchanger 240 if present. During the first step of the drying process, a first air flow is discharged out of the first dryer 231. A heat exchanger 260 (e.g. a heat pump 260) is used to capture the heat from the first air flow and heat a second air flow. In a second step of the drying process, the thermophilic fermented culture is further dried using the second air flow. Additionally, the drying process may comprise more than two steps, each consequent step may use a heat exchanger (such as the heat exchanger 260) to capture heat from an air flow discharged in a previous dryer and heat an air flow for further drying the thermophilic fermented culture.

As described, heat produced during aerobic fermentation is used to heat up an air flow for drying an aerobically fermented culture. By using said heat, extra energy sources are avoided, as the ones coming from fossil fuel resources. Consequently, the invention provides a more environmental friendly process and system for obtaining a protein culture from aerobic fermentation. Furthermore, since heat produced during aerobic fermentation is more than enough to heat the air flow used during drying, side processes, as a CIP process, can also take advantage of this heat production during aerobic fermentation such that part of the heat produced can further be used to heat cleaning substances in the CIP process thereby, reducing or even avoiding the further use of extra source of energy.

Although the invention has been described as drying (thermal drying) the fermented culture after fermentation using a heated air flow. Since thermal drying (or thermal dehydration) is more expensive that mechanical drying (or mechanical dehydration) in terms of energy cost, the drying stage is optionally performed in two stages: a fist stage of mechanical drying and a second stage of thermal drying.

In the first stage, after fermentation, the thermophilic fermented culture is dried by using at least one of sieving, filtration, decantation and decanter centrifugation, such that dry matter concentration of the thermophilic fermented culture is at least 8%, 10%, 12% in weight. Optionally, the fermented culture can be pasteurized. By pasteurizing, thermophilic organisms presents in the fermented culture are thermally inactivated (or merely inactivated) allowing to extend storage life of the fermented culture as well as killing harmful microbes in fermented culture without affecting its nutritional value. Then, concentrated, and optionally pasteurized, fermented culture is dried by pressing residual water out using e.g. compressed air, as a pneumapress, and/or mechanical pressing, as e.g. a filter press, a belt press or a screw press to obtain a biomass (cake), such that dry matter concentration of the fermented culture is at least 20%, 25%, 30%, 35%, 40%, 45%, 50% in weight. The Optionally, the water fraction that is obtained after sieving, filtering, decanting, centrifugating and/or pressing the fermented culture is recycled back to the fermentation and/or used for further fermentation batches.

After pressing the fermented culture to a cake, optionally, the cake can be milled or extruded e.g. to enable drying, preferably air drying. Preferably, the particle size of the cake is reduced by physical means to enable (more efficient) drying of the cake. This can optionally done by extrusion of the cake through holes with a diameter of 0.6, 0.7, 0.8, 0.9, 1.0, 1.2, 1.4, 1.6, 1.8 2, 3, 4, 5, 6, 7, 8, 9 mm or even up to 10 mm, using extruders that are known in the art per se. If however the dry matter concentration of the cake after pressing is so high, that extrusion of the cake is no longer possible (e.g. when the cake is too firm to allow for extrusion), the particle size of the cake can be reduced by a combination of milling and sieving. As a milling step any type of mill known in the art per se can be used, such as e.g. a knife mill or a hammer mill, etc. To obtain homogeneous particle size of the milled cake, the larger particles still present after milling can be removed before drying by sieving with a pore diameter size in the sieve of 0.5, 1.0, 1.2, 1.4, 1.6, 1.8, 2.0, 3, 4, 5, 6, 7, 8, 9 or even 10 mm. The resulting milled cake would have preferably a particle size between 1-9 mm before drying, more preferable a particle size of 8 mm before drying. By reducing the particle size, evaporation of water from the cake is more efficient and faster.

In the second stage, the pressed fermented culture (or cake) is dried using heat produced during the aerobic fermentation. The hot air can dry the cake in a gentle and cost effective way in e.g. a belt dryer or fluid bed dryer.

FIG. 6 shows an exemplary representation of a drying process using a horizontal fluidized bed dryer. The dryer can be the dryer 230 of FIGS. 2-4. In FIG. 6, the dryer 230 is a horizontal fluidized bed dryer. The dryer 230 comprises a first dryer 231, a second dryer 232, a third dryer 233 and a fourth dryer 234. A pressed fermented culture (or cake) is continuously supplied from an inlet of the first drying chamber 231 and dried while it moves horizontally to an outlet 234a of the fourth drying chamber 234 over a grid 280 through which heated air flow is blown upwardly. via an inlet, into corresponding drying chamber. The cake continuously moving horizontally from the first drier 231 to the fourth drier 234 is commonly known as a bed 290.

Each drying chamber comprises an outlet through which air flow is discharged out of the drying chamber. The discharged air flow can be the first air flow of FIG. 5. A heat exchanger (e.g. the heat pump 260 of FIG. 5) captures the heat from the first air flow and heats a second air flow which is then blown upwardly into the next drying chamber. The heated air flow can be the second air flow of FIG. 5.

During drying, bubbles are formed in the bed 290. Bubbles in fluidized beds are generally beneficial as they promote solids mixing, heat transfer and mass transfer. However, bubbles tend to coalesce and migrate towards the center of the dryer. A baffle 270 is placed between each dryer in order to improve the bubbles distribution over the bed cross-section. Various types of baffle can be used, for example, wire mesh, perforated plate, turn plate, louver plate, and ring. During drying, agglomeration and deposits near baffles, which may disturb the uniform bed fluidization and cause local overheating of the cake Hence, velocity of particles within the bed can be limited by applying vibration to the bed. Vibration limits agglomeration and deposits near baffles.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” 15 thus usually means “at least one”.

In view of the above, the present invention can now be summarised by the following embodiments:
Embodiment 1. A process (100) for reusing heat in a protein culture, the process comprising the steps of: aerobically fermenting (110) a material with an thermophilic organism to provide a thermophilic fermented culture; and performing a drying process (120) on the thermophilic fermented culture using heat produced during the aerobic fermentation.
Embodiment 2. The process of embodiment 1, wherein the process comprises using a first heat exchanger to capture the heat produced during the aerobic fermentation process and heating an air flow for the drying process.
Embodiment 3. The process of embodiment 2, wherein the first heat exchanger is a heat pump.
Embodiment 4. The process of embodiment 3, wherein the first heat pump uses ammonia or R1234 as a refrigerant.
Embodiment 5. The process of embodiment 2-3, wherein capturing the heat produced during fermentation comprises capturing heat from a gas flow discharged during the fermentation, wherein the gas flow has a temperature of 36 C or more and a relative humidity of 100%.
Embodiment 6. The process of any embodiment 2-5, wherein the drying process comprises: further increasing the temperature of the air flow flowing out of the first heat exchanger using a second heat exchanger.
Embodiment 7. The process of embodiment 6, wherein the second heat exchanger is a steam heat exchanger.
Embodiment 8. The process of embodiment 2-7, wherein the heated air flow used during drying has a temperature between 70 C and 120 C.
Embodiment 9. The process of embodiment 2-8, wherein the drying process comprises: drying, in a first step, the thermophilic fermented culture using the heated air flow, and drying, in a second step, the thermophilic fermented culture using heat produced during the first step of drying.
Embodiment 10. The process of embodiment 9, wherein using the heat produced during the first step of drying comprises using a third heat exchanger to capture the heat from a first air flow discharged during the first step of drying and heating a second air flow.
Embodiment 11. The process of embodiment 10, wherein the third heat exchanger is a heat pump.
Embodiment 12. The process of any preceding embodiment, further comprises: using waste heat produced during the aerobic fermentation in a clean-in-process, CIP.
Embodiment 13. The process of any preceding embodiment, wherein aerobically fermenting a material with an thermophilic organism to provide a thermophilic fermented culture comprises: introducing sterile or unsterile air.
Embodiment 14. The process of embodiment 13, wherein the sterile or unsterile air is introduced at a predetermined air flow rate.
Embodiment 15. The process of any preceding embodiment, wherein aerobically fermenting a material with an thermophilic organism to provide a thermophilic fermented culture comprises: introducing at least one substance to control pH.
Embodiment 16. A system (200) for reusing heat in a protein culture, the system comprising: a fermenter (210) configured to: aerobically ferment a material with an thermophilic organism to provide a thermophilic fermented culture, and discharge a gas flow out of the fermenter; a dryer (230) configured to: dry the thermophilic fermented culture to recover a protein culture; and a first heat exchanger (220) configured to: capture heat from the discharged gas flow of the fermenter and use the captured heat to provide a heated air flow for the dryer.
Embodiment 17. The system of embodiment 16, wherein the fermenter (210) comprises an air inlet (211) through which introducing sterile or unsterile air.
Embodiment 18. The system of embodiment 17, wherein the sterile or unsterile air is introduced at a predetermined air flow rate.
Embodiment 19. The system of embodiment 16-18, wherein the fermenter (210) comprises an air inlet (211) through which introducing at least one substance to control pH.
Embodiment 20. The system of embodiment 16-19, wherein the discharged gas flow has a temperature of 36 C or more and a relative humidity of 100%.
Embodiment 21. The system of embodiment 16-20, wherein the system further comprises: a second heat exchanger (240) configured to further increase the temperature of the heated air flow flowing out of the first heat exchanger (220).
Embodiment 22. The process of embodiment 16-21, wherein the dryer (230) comprises: a first dryer (231) configured to dry the thermophilic fermented culture by using the heated air flow; a third heat exchanger (260) configured to capture heat from a first air flow discharged out of the first dryer (231) and use the captured heat to provide a heated second air flow; and a second dryer (232) configured to dry the thermophilic fermented culture to recover a protein culture by using the second air flow.
Embodiment 23. The process of embodiment 22, wherein the third heat exchanger is a heat pump.
Embodiment 24. The system of embodiment 16-23, wherein the system further comprises: an additional condenser (250) configured to heat up water.
Embodiment 25. The system of embodiment 16-24, wherein the heated air flow used during drying has a temperature between 70 C and 120 C.
Embodiment 26. The system of embodiment 16-25, further comprises: a clean-in-process, CIP, system configured to use heat produced during the aerobic fermentation.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiments disclosed, but that the invention will include all embodiments falling within the scope of the appended claims.

1. A process for reusing heat in a protein culture, the process comprising the steps of:
aerobically fermenting a material with an thermophilic organism to provide a thermophilic fermented culture; and
performing a drying process on the thermophilic fermented culture using heat produced during the aerobic fermentation,
wherein the process comprises using a first heat exchanger to capture the heat from a gas flow discharged during the aerobic fermentation and heating an air flow for a drying process.

2. The process of claim 1, wherein the first heat exchanger is a heat pump.

3. The process of claim 2, wherein the first heat pump uses ammonia or R1234 as a refrigerant.

4. The process of claim 2-32, wherein the gas flow has a temperature of 36 C or more and a relative humidity of 100%.

5. The process of any preceding claim 1, wherein the drying process comprises:
further increasing the temperature of the air flow flowing out of the first heat exchanger using a second heat exchanger.

6. The process of claim 5, wherein the second heat exchanger is a steam heat exchanger.

7. The process of claim 1, wherein the heated air flow used during drying has a temperature between 70 C and 120 C.

8. The process of claim 1, wherein the drying process comprises:
drying, in a first step, the thermophilic fermented culture using the heated air flow, and
drying, in a second step, the thermophilic fermented culture using heat produced during the first step of drying.

9. The process of claim 8, wherein using the heat produced during the first step of drying comprises using a third heat exchanger to capture the heat from a first air flow discharged during the first step of drying and heating a second air flow.

10. (canceled)

11. The process of claim 1, further comprising:
using waste heat produced during the aerobic fermentation for increasing a temperature of a cleaning substance used in a clean-in-process, CIP.

12. The process of claim 1, wherein aerobically fermenting a material with an thermophilic organism to provide a thermophilic fermented culture comprises:
introducing sterile or unsterile air.

13. The process of claim 12, wherein the sterile or unsterile air is introduced at a predetermined air flow rate.

14. The process of any preceding claim 1, wherein aerobically fermenting a material with an thermophilic organism to provide a thermophilic fermented culture comprises:
introducing at least one substance to control pH.

15. A system for reusing heat in a protein culture, the system comprising:
a fermenter configured to:
aerobically ferment a material with an thermophilic organism to provide a thermophilic fermented culture, and
discharge a gas flow out of the fermenter;
a dryer configured to:
dry the thermophilic fermented culture to recover a protein culture; and
a first heat exchanger configured to:
capture heat from the discharged gas flow of the fermenter and use the captured heat to provide a heated air flow for the dryer.

16. The system of claim 15, wherein the fermenter comprises an air inlet for introducing sterile or unsterile air and/or introducing at least one substance to control pH.

17. (canceled)

18. The system of claim 15, further comprising:
a second heat exchanger configured to further increase the temperature of the heated air flow flowing out of the first heat exchanger.

19. The system of claim 15, wherein the dryer comprises:
a first dryer configured to dry the thermophilic fermented culture by using the heated air flow;
a third heat exchanger configured to capture heat from a first air flow discharged out of the first dryer and use the captured heat to provide a heated second air flow; and
a second dryer configured to dry the thermophilic fermented culture to recover a protein culture by using the second air flow.

20. The system of claim 19, wherein the third heat exchanger is a heat pump.

21. The system of claim 2015, further comprising:
an additional condenser configured to heat up water.

22. (canceled)

23. The system of claim 15, further comprises:
a clean-in-process, CIP, system configured to use heat produced during the aerobic fermentation for increasing a temperature of a cleaning substance.
